(19) United States
(12) Patent Application Publication
(10) Pub. No.: US 2024/0423750 A1
(43) Pub. Date: Dec. 26, 2024

(54) SURGICAL PLANNING AND DISPLAY

(71) Applicant: AUGMEDICS LTD., Yokneam Illit (IL)

(72) Inventors: Nissan Elimelech, Be'erotaim (IL); Monica Marie Kuhnert, Chicago, IL (US); Gal Bar-Zohar, Yokneam Hamoshava (IL); Lilach Meidan, Zichron Yaacov (IL); Stuart Wolf, Yokneam (IL)

(21) Appl. No.: 18/693,338

(22) PCT Filed: Sep. 23, 2022
(86) PCT No.: PCT/IB2022/059030

§ 371 Date: Mar. 19, 2024

Related U.S. Application Data
(60) Provisional application No. 63/248,487, filed on Sep. 26, 2021.

(51) Int. Cl. A61B 90/00; A61B 17/56; A61B 17/70; A61B 34/00; A61B 34/10; A61B 90/50; G06N 3/08

(52) U.S. Cl. CPC A61B 90/37; A61B 17/7076; A61B 34/10; G06N 3/08; A61B 2017/564; A61B 2034/107; A61B 2034/256; A61B 2090/365; A61B 2090/367; A61B 2090/3762; A61B 2090/502

(57) ABSTRACT

A method includes accessing a computerized tomography (CT) scan of a human subject and defining a first two-dimensional (2D) slice (200) of the scan and a second 2D slice (204) of the scan, so that the first and the second slices intersect in an intersection line (224) defining a desired trajectory. The method further includes overlaying on the intersection line an icon (250) representing an object used in a procedure on the human subject, and rendering a three-dimensional (3D) image (650) of the human subject, incorporating the overlayed icon, from the CT scan.

### CROSS-REFERENCE TO RELATED APPLICATION

This application claims the benefit of U.S. Provisional Patent Application 63/248,487, filed Sep. 26, 2021.

### FIELD

The present disclosure relates generally to systems and methods for surgery, and specifically to systems and methods to facilitate image-guided surgery or other medical intervention.

### BACKGROUND

Image-guided surgery (IGS) is a surgical procedure where a medical professional may use surgical instruments that are tracked, together with images that are presented to the professional, to assist the professional in performing the procedure. In augmented reality IGS the images may be presented to the professional overlaid on his view of the scene, for example, e.g., in a head-set worn by the professional, and are typically presented in real time. During a spinal surgery procedure, for example, the images presented may show elements, such as vertebrae and/or inserts to the vertebrae, that are not directly visible to the professional. However, because the elements are not directly visible during the procedure. the element images cannot be acquired by a camera.

### SUMMARY

An embodiment of the present disclosure provides a method including:

accessing a computerized tomography (CT) scan of a human subject:

defining a first two-dimensional (2D) slice of the scan and a second 2D slice of the scan, so that the first and the second slices intersect in an intersection line defining a desired trajectory;

overlaying on the intersection line an icon representing an object used in a procedure on the human subject; and

rendering a three-dimensional (3D) image of the human subject, incorporating the overlayed icon, from the CT scan.

The object may include at least one of a screw and a planned trajectory.

The planned trajectory may include a direction for drilling into the human subject.

The icon of the planned trajectory may consist of an icon termination point and an icon initial point respectively corresponding to a planned trajectory termination point and a planned trajectory initial point.

In a disclosed embodiment the planned trajectory initial point includes a skin incision point, and the planned trajectory termination point includes a drill end point.

In another disclosed embodiment the icon of the screw includes an icon termination point and an icon initial point respectively corresponding to a screw tip and a screw head.

In yet another disclosed embodiment the object includes a screw configured to be inserted along the desired trajectory into a selected vertebra of the human subject.

The 3D image may include at least one further icon representing at least one further screw configured to be inserted along respective at least one selected trajectory, parallel to the desired trajectory, into respective at least one vertebrae proximate to the selected vertebra.

The 3D image may include a rod icon representing a rod joining respective heads of the screws and the at least one further screw.

The 3D image may include a further icon representing a further screw configured to be inserted along a selected trajectory, different from the desired trajectory, into the selected vertebra.

The method may further include, during the procedure, presenting the 3D image on an augmented reality display, while aligning the 3D image with a view through the display of the human subject and the object.

There is further provided, according to an embodiment of the present disclosure, a method including:

assembling a corpus of data sets of respective procedures performed on human subjects, each data set including, for a given human subject, a computerized tomography (CT) scan thereof, an identification of a vertebra therein wherein at least one screw has been inserted, and data descriptive of the at least one screw;

training an artificial neural network (ANN) using the corpus of data sets;

receiving a further CT scan from a further human subject for the trained ANN; and

rendering a three-dimensional (3D) image of the further human subject, in response to an output of the trained ANN, the 3D image including a representation of a further human subject vertebra and of a further screw inserted therein.

In a disclosed embodiment the at least one screw includes a single screw, and each data set includes a further identification of at least one further vertebrae, wherein at least one further screw has been inserted, and further data descriptive of the at least one further screw, and a rod identification of a rod joining the single screw and the at least one further screw,

wherein the 3D image includes, in response to the output of the trained ANN, a rod representation of a rod joining the further screw and at least one additional screw inserted into respective vertebrae of the human subject.

There is further provided, according to an embodiment of the present disclosure, apparatus including:

a screen, configured to present a first two-dimensional (2D) slice of a computerized tomography (CT) scan of a human subject and a second 2D slice of the scan, so that the first and the second slices intersect in an intersection line defining a desired trajectory; and

a processor, configured to:

overlay on the intersection line an icon representing an object used in a procedure on the human subject; and

render a three-dimensional (3D) image of the human subject, incorporating the overlayed icon, from the CT scan.

There is further provided, according to an embodiment of the present disclosure, apparatus including:

a display; and

a processor configured to:

assemble a corpus of data sets of respective procedures performed on human subjects, each data set including, for a given human subject, a computerized tomography (CT) scan thereof, an identification of a vertebra therein wherein at least one screw has been inserted, and data descriptive of the at least one screw;

train an artificial neural network (ANN) using the corpus of data sets;

input a further CT scan from a further human subject into the trained ANN;

render a three-dimensional (3D) image of the further human subject, in response to an output of the trained ANN, the 3D image including a representation of a further human subject vertebra and of a further screw inserted therein; and

present the 3D image on the display.

There is further provided, according to an embodiment of the present disclosure, a method for planning image-guided surgery of a human subject, including:

defining a plurality of two-dimensional (2D) slices of a computerized tomography (CT), wherein a first slice and a second slice of the plurality of 2D slices intersect in an intersection line, the intersection line defining a desired trajectory for the image-guided surgery;

overlaying an icon on the intersection line, the icon representing an object used in a procedure on the human subject;

rendering a three-dimensional (3D) image of the human subject from the CT scan that incorporates the icon; and

presenting the 3D image on an augmented reality display, wherein the 3D image is aligned with a view through the augmented reality display of the human subject and the object.

There is further provided, according to an embodiment of the present disclosure, an apparatus for planning image-guided surgery of a human subject, including:

a head mounted display (HMD);

a display configured to present a plurality of two-dimensional (2D) slices of a computerized tomography (CT) scan of the human subject, a first slice and a second slice of the plurality of 2D slices intersecting in an intersection line defining a desired trajectory; and

a processor, configured to:

overlay an icon on the intersection line, the icon representing an object used in a procedure on the human subject;

render a three-dimensional (3D) image of the human subject from the CT scan that incorporates the icon; and

present the 3D image on an augmented reality display, wherein the 3D image is aligned with a view through the augmented reality display of the human subject and the object.

The object may include at least one of a screw and a planned trajectory.

The planned trajectory may consist of a direction for drilling into the human subject.

In a disclosed embodiment the icon of the planned trajectory consists of an icon termination point and an icon initial point respectively corresponding to a planned trajectory termination point and a planned trajectory initial point.

In a further disclosed embodiment the planned trajectory initial point includes a skin incision point, and the planned trajectory termination point includes a drill end point.

In a yet further disclosed embodiment the icon of the screw consists of an icon termination point and an icon initial point respectively corresponding to a screw tip and a screw head.

In another disclosed embodiment the object includes a screw configured to be inserted along the desired trajectory into a selected vertebra of the human subject.

In an alternative embodiment the 3D image includes at least one further icon representing at least one further screw configured to be inserted along respective at least one selected trajectories, parallel to the desired trajectory, into respective at least one vertebrae proximate to the selected vertebra.

In a further alternative embodiment the 3D image includes a rod icon representing a rod joining respective heads of the screw and the at least one further screw.

In a yet further alternative embodiment the 3D image includes a further icon representing a further screw configured to be inserted along a selected trajectory, different from the desired trajectory, into the selected vertebra.

The augmented reality retaining structure may be spectacles.

The augmented reality retaining structure may be glasses.

The augmented reality retaining structure may be a head mounted display (HMD).

There is further provided, according to an embodiment of the present disclosure, a method including:

accessing a three dimensional (3D) anatomy scan of a human subject;

defining a first two-dimensional (2D) view the scan, a second 2D view of the scan, and a third 2D view of the scan to provide an initial view of an area of interest of the human subject, wherein the first, second and third 2D views are generated from the scan; and

rotating at least one of the first, second, and third 2D views so as to provide an improved view of the area of interest.

In a disclosed embodiment the first, second and third 2D views define respective first, second and third normals thereto, and rotating the at least one of the first, second, and third 2D views consists of rotating the first view about one of the second normal and the third normal.

In a further disclosed embodiment the first, second and third 2D views define respective first, second and third normals thereto, and rotating the at least one of the first, second, and third 2D views consists of rotating the first view about the second normal and the third normal.

In a yet further disclosed embodiment the 2D views are 2D slices of the scan.

In another disclosed embodiment the 2D views are axial, sagittal, and coronal views of the human subject.

In an alternative embodiment the 3D anatomy scan is a Computerized Tomography (CT) scan.

In another alternative embodiment at least one of the first 2D view the scan, the second 2D view of the scan, and the third 2D view of the scan is a digitally reconstructed radiograph (DRR).

In yet another alternative embodiment the method further includes translating at least one of the first, second, and third 2D views so as to provide an improved view of the area of interest.

The rotating may be performed following a user instruction.

In another embodiment the first, second and third 2D views define respective first, second and third normals, and each 2D view of the first, second, and third 2D views may be rotated with respect to only one other normal of the first, second and third normals.

There is further provided, according to an embodiment of the present disclosure, a system as described and illustrated herein.

There is further provided, according to an embodiment of the present disclosure, a method as described and illustrated herein.

For purposes of summarizing the disclosure, certain aspects, advantages, and novel features are discussed herein. It is to be understood that not necessarily all such aspects, advantages, or features will be embodied in any particular embodiment of the disclosure, and an artisan would recognize from the disclosure herein a myriad of combinations of such aspects, advantages, or features.

The present disclosure will be more fully understood from the following detailed description of the embodiments thereof, taken together with the drawings, in which:

### BRIEF DESCRIPTION OF THE DRAWINGS

Non-limiting features of some embodiments of the invention are set forth with particularity in the claims that follow. The following drawings are for illustrative purposes only and show non-limiting embodiments. Features from different figures may be combined in several embodiments.

FIG. 1 is a schematic illustration of a surgical planning and display system, according to an embodiment of the present disclosure;

FIG. 2 is a schematic illustration of an augmented reality assembly of the system, according to an embodiment of the present disclosure;

FIG. 3 is a flowchart describing steps performed in a planning stage of a surgical procedure, according to an embodiment of the present disclosure;

FIGS. 4A-8 are schematic drawings illustrating some of the steps of the flowchart of FIG. 3, according to an embodiment of the present disclosure;

FIG. 9 is a schematic diagram of an artificial neural network used in the planning stage of the surgical procedure, according to an alternative embodiment of the present disclosure;

FIG. 10A is a flowchart of steps describing how the artificial neural network is trained and FIG. 10B is a flowchart of how the network is used, according to an embodiment of the present disclosure;

FIGS. 11A and 11B are schematic diagrams of images of a patient presented to a professional, according to an embodiment of the present disclosure;

FIG. 12 is a schematic figure illustrating an exemplary head-mounted display, according to an embodiment of the present disclosure;

FIG. 13 is a schematic view of a section of a patient's spine including a planned screw placement, according to an embodiment of the present disclosure;

FIG. 14 is a schematic view of the back of the patient of FIG. 13 including a planned incision for inserting the screw of FIG. 13; and

FIG. 15 is a schematic view of a planning stage of a procedure involving a bone cut, according to an embodiment of the present disclosure.

### DETAILED DESCRIPTION

### Overview

Embodiments of the present disclosure provide a software tool that enables a medical professional to use a work station to generate and store images, e.g., two dimensional (2D) or three-dimensional (3D) images, rendering or models of the anatomy of a patient for use during performance of a medical procedure on the patient. During the procedure, the professional can wear a head mounted display (HMD) which registers and tracks the patient in a frame of reference. e.g. of the HMD or any other determined frame of reference. By virtue of the registration and tracking, a processor of, for example, the HMD uses a 3D image, e.g., based on a 3D image captured by a three-dimensional modality (such as Computerized Tomography (CT) device) or rendered from 2D images, that is aligned with a scene viewed by the professional, and that is overlayed on the display of the HMD in an augmented reality manner. The work station may be various types of client computers including desktop computers, notebook computers. handheld computers or the like.

As described herein, the software tool enables the professional to generate the images in the work station. Although, for simplicity, in the following description the procedure referred to is a spinal procedure. one having ordinary skill in the art will be able to modify the description, mutatis mutandis, for other surgical procedures such as, for example, those on hip bones, pelvic bones, leg bones, arm bones, ankle bones, foot bones, shoulder bones, cranial bones, oral and maxillofacial bones, or sacroiliac joints.

In certain embodiments, the software tool uses a computerized tomography (CT) image, typically a DICOM (Digital Imaging and Communications in Medicine) file, of the spine of the patient, and initially presents a plurality of 2D images (e.g., two images, three images, four images, etc.), also herein termed slices, of the spine on the work station. In some embodiments, other views, such as x-ray views, based e.g., based on DRR (digitally reconstructed radiograph) images generated from the CT image, may also be presented. In certain embodiments, the initial slices presented are an axial view, a sagittal view. and a coronal view. As is described below, the professional is able to manipulate, i.e., rotate and/or translate, one or more of the slices independently. Consequently, rather than continuing to use the terms axial, sagittal, and coronal for the embodiment that presents three slices, the following description uses the terms a-slice, s-slice, and c-slice. Each slice is a plane, and there is a normal to each of the slices, herein respectively termed an a-normal, an s-normal, and a c-normal to the a-slice, the s-slice, and the c-slice.

In certain embodiments, the slices intersect each other (in the initial axial/sagittal/coronal view case the intersections are mutually orthogonal). Thus, the a-slice is intersected by the s-slice and the c-slice, and the two lines of intersection are displayed on the a-slice view. (The other two slice views each have two lines of intersection displayed.) In certain embodiments, handles are attached to each of the intersecting lines, and the handles are configured to enable the slice of the intersecting line to be translated and/or rotated.

For example, in the a-slice display, the handles of the s-slice intersection can be used to translate the s-slice in any direction parallel to the a-slice, and/or to rotate the s-slice around the a-normal. Any such manipulation of the s-slice will be apparent in the s-slice view, as well as in changes in the intersecting lines in the s-slice and/or the c-slice views.

The three slices intersect at one point, and each pair of slices intersect at a line. Thus, since the slices are independently manipulable, the professional can direct the intersection point to any location in the 3D image file. Similarly, any line may be defined, as the intersection of a pair of slices, each of which has been selected and manipulated.

For example, prior to a procedure involving screw placement in a vertebra of a patient, the professional may manipulate the three image slices to intersect at the point on the vertebra where the screw tip is to enter, and also manipulate two of the slices so that their intersection line corresponds to the desired screw trajectory. An icon having details of the screw (e.g., the screw head and length) can be added to the image slices, with the screw tip of the icon being at the intersection point, and the icon of the screw lying along the intersection line, and the composite images saved. Other objects, such as another trajectory used for drilling, e.g., on a bony structure near a screw top, may be generated and saved.

As stated above, saved 2D images may be rendered to provide a 3D image. During the procedure, when the professional is to insert the screw, the 3D image can be recalled. Since the HMD is tracking the patient, a processor of the HMD, for example, is able to use the recalled image to present, on the augmented reality display of the HMD, a three-dimensional (3D) image of the screw and vertebra that is registered with the actual vertebra, thus assisting the professional in positioning the actual screw. In some embodiments, the registered image may be presented, additionally or alternatively, on another display, such as a display of the work station. More detail of this procedure, together with examples of other procedures using saved images for the augmented reality display, are provided in the following System Description section.

Several embodiments are particularly advantageous because they include the benefits of assisting in screw positioning, as stated above, as well as enabling the professional to check, and if necessary alter, dimensions of screws and/or rods to be used in a planned procedure, as is described further below.

### System Description

In the following, all directional references (e.g., upper, lower, upward, downward, left, right, top, bottom, above, below, vertical, and horizontal) are only used for identification purposes to aid the reader's understanding of the present disclosure, and do not create limitations, particularly as to the position, orientation, or use of embodiments of the disclosure.

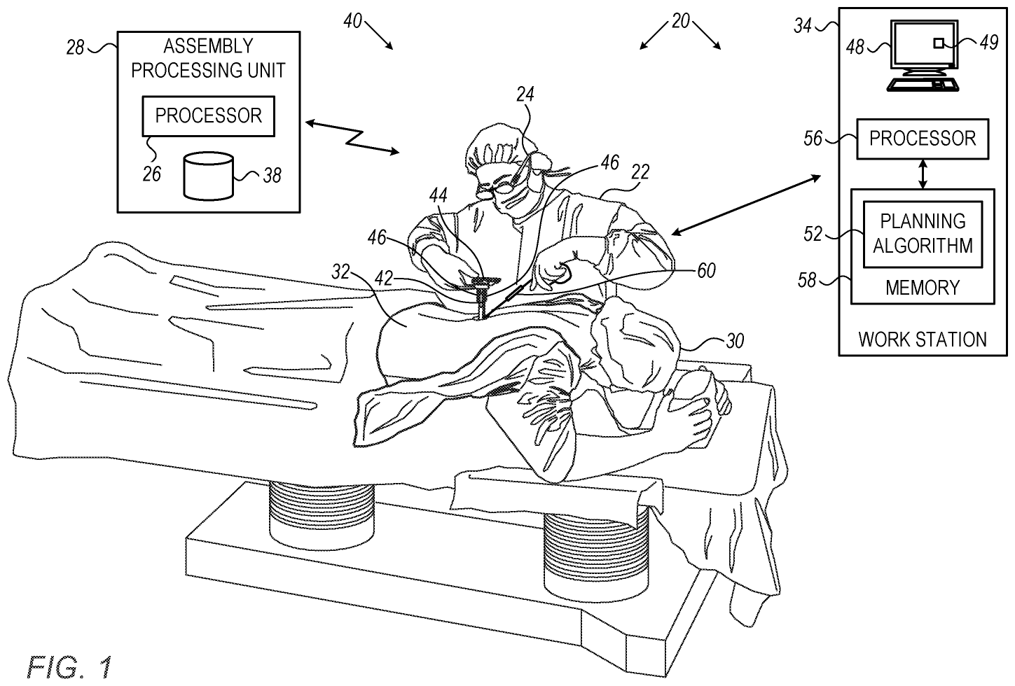

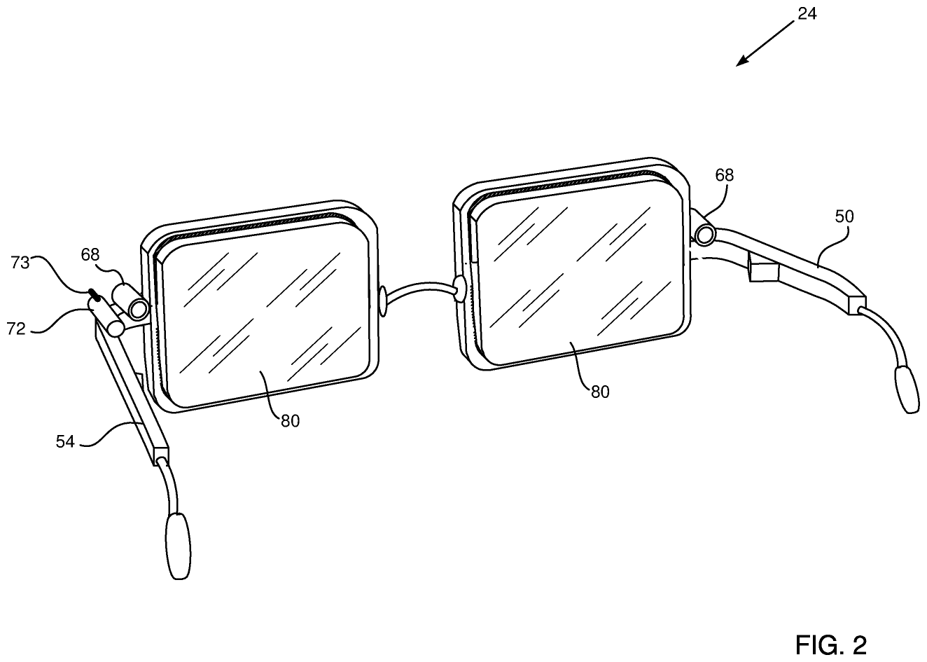

Reference is now made to FIG. 1, which is a schematic illustration of a surgical planning and display system 20, and to FIG. 2, which is a schematic illustration of an augmented reality assembly 24 of the system, according to an embodiment of the present disclosure. (The description of system 20 herein also includes a description of a navigation system, and those having ordinary skill in the art will be able to adapt the description, mutatis mutandis, to describe the navigation system.) System 20 and assembly 24 are used by a professional 22 to assist the professional in performing a surgical procedure, herein by way of example assumed to comprise a spinal procedure wherein a screw 60 having known dimensions is inserted into a vertebra of the spine of a patient 30. During the procedure, the professional 22 can wear an augmented reality assembly 24. In certain embodiments, the assembly is controlled by an assembly processing unit 28. The assembly processing unit 28 comprises a processor 26, which operates and communicates with elements of the system 20. During a planning stage of the system 20, prior to performing the illustrated surgical procedure, the professional 22 uses a work station or other client computer 34 that has a display or screen 48 and a user interface 49. In certain embodiments, the work station 34 is operated by a work station processor 56 that communicates with a memory 58 wherein is stored a planning algorithm 52. The processor 56 can access the planning algorithm 52 to generate material that may be used by the augmented reality assembly 24 and the assembly processing unit 28 during the surgical procedure. The assembly processing unit 28. any of its components (e.g., processor 26 or database 38) or a combination of its components may be mounted on the augmented reality assembly 24 or on the HMD or on work station 34. The planning stage is explained below.

The processor 26 of the assembly processing unit 28 is able to access a database 38. In certain embodiments, stored on the database 38 are images derived from the work station 34, other visual elements, and/or any other types of data, including computer code, used by the augmented reality assembly 24. In certain embodiment, database 38 is Software for the augmented reality assembly 24 or work station 34 (or both) may be downloaded to the database 38 or to the work station 34 in electronic form, over a network, for example. Alternatively or additionally, the software may be provided on non-transitory tangible media, such as optical, magnetic, or electronic storage media.

In certain embodiments, during an initial stage of the surgical procedure the professional 22 mounts an anchoring device, such as a clamp or a pin, to a bone or bones of the patient. For example, the professional 22 can make an incision into a patient's back 32. The professional 22 may then insert a spinous process clamp 42, into the incision, so that opposing jaws of the clamp 42 are located on opposite sides of a spinous process. The professional 22 adjusts the clamp 42 to grip one or more spinous processes, selected by the professional 22, of the patient.

It will be understood that embodiments of the disclosure described herein are not limited to the use of a clamp, and are also not limited to the tracking method and registration system described herein.

In certain embodiments, the professional 22 attaches an alignment target 44 to a base 46 of the clamp 42 (or any other bone anchoring device used). The target 44, when attached to the base 46, can operate as a patient marker 40. The patient marker 40 thus comprises the alignment target 44 coupled to the base 46. As is described below, the patient marker 40 can be used by the augmented reality assembly 24 to determine the position and orientation of the patient 30, in a frame of reference defined by the augmented reality assembly 24, during the surgical procedure. The position and orientation of the patient 30 is determined with respect to a tracking system tracking the patient marker 40. In some embodiments the tracking system is mounted on or included in the augmented reality assembly 24.

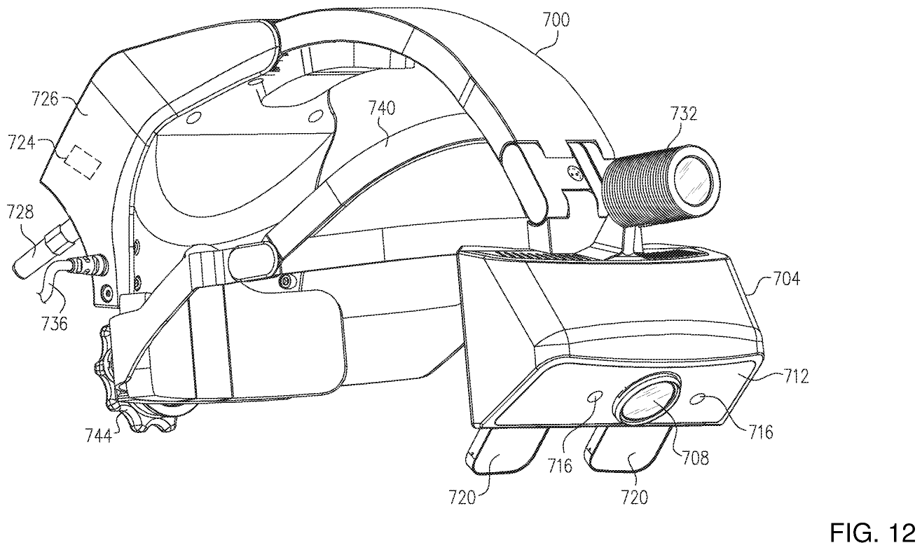

While the augmented reality assembly 24 may be incorporated for wearing into a number of different retaining structures on the professional 22, in the embodiment illustrated in FIG. 1 the retaining structure is assumed to be similar to a pair of spectacles. An alternative retaining structure, comprising a head mounted display (HMD) that is integrated into a helmet worn by the professional 22, is described below, with reference to FIG. 12. Those having ordinary skill in the augmented reality art will be aware of other possible retaining structures that may be worn by the professional 22, and all such structures are assumed to be comprised within the scope of the present disclosure.

As illustrated in FIG. 2, the augmented reality assembly 24 is configured as a pair of spectacles 50 mounted on a frame 54. Each lens of the spectacles 50 comprises an augmented reality display 80, the displays allowing the professional 22 to view entities, such as part or all of the patient 30 through the displays 80, and which are also configured to present to the professional 22 images that may be received, e.g., from the database 38, as well as other images generated, e.g., by the processor 26 and that are described herein below. In certain embodiments. at least a portion of display 80 is transparent, substantially transparent or at least partially transparent. In certain embodiments, each of display 80 includes a display area. The presented images overlaid on the scene viewed by the professional may be projected onto the display area. The display may be opaque, substantially opaque, partially transparent, or substantially transparent or transparent. The portion of displays 80, which is not the display area, e.g., the portion of each display 80 which surrounds the display area, is transparent or substantially transparent.

In certain embodiments, the augmented reality assembly 24 comprises at least one image capturing device 68. In the embodiment illustrated in FIG. 2 there are two such devices. In certain embodiments, each device 68 comprises a camera configured to capture images of scenes viewed by the professional's eyes. including images of the patient marker 40, in the visible spectrum.

In certain embodiments, the augmented reality assembly 24 comprises an image capturing device 72, also herein termed a camera 72. In certain embodiments, the camera 72 is configured to capture images of elements of a scene, including the patient marker 40, in front of the augmented reality assembly 24, that are produced from radiation projected by a projector 73. In certain embodiments, the camera 72 and the projector 73 operate in a non-visible region of the spectrum, such as in, for example, the near infra-red spectrum. The projector 73 can be located in close proximity to the camera 72, so that radiation from the projector 73, that has been retroreflected, is captured by the camera 72. The camera 72 can comprise a bandpass filter configured to block other radiation. such as that projected by surgical lighting.

The arrangement of elements of assembly 24 illustrated in FIG. 2 is by way of example. and other arrangements of the elements, such as having device 68, camera 72, and/or projector 73 between or above displays 80, are within the scope of the present disclosure.

At least some retroreflected radiation is typically received from the patient marker 40, and the processor 26 may use the image of the patient marker 40 produced by camera 72 from the received radiation to track the patient marker 40, and thus the position and orientation of the patient 30 in the frame of reference of the augmented reality assembly 24 (to which the camera 72 and the projector 73 can be attached).

As is described below, embodiments of the disclosure form two-dimensional (2D) images of the patient 30 from a computerized tomography (CT) scan of the patient. (The 2D images can be generated in the planning stage of system 20 referred to above.) By tracking the position and orientation of the patient 30. the processor 26 is able to present. on the displays 80, three-dimensional (3D) images of the patient 30, including 3D images derived from the 2D images, that are correctly registered with the physician's actual view of the patient 30. In certain embodiments, the 3D images are presented to the professional 22 during the surgical procedure described herein.

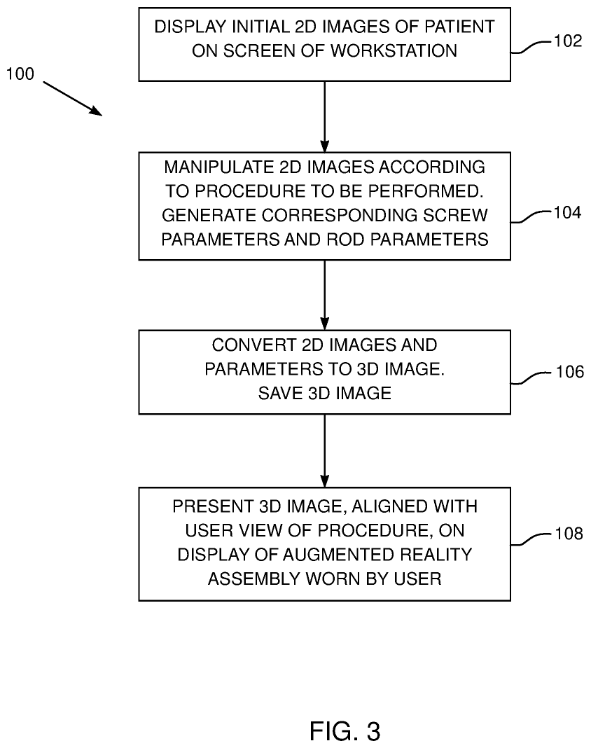

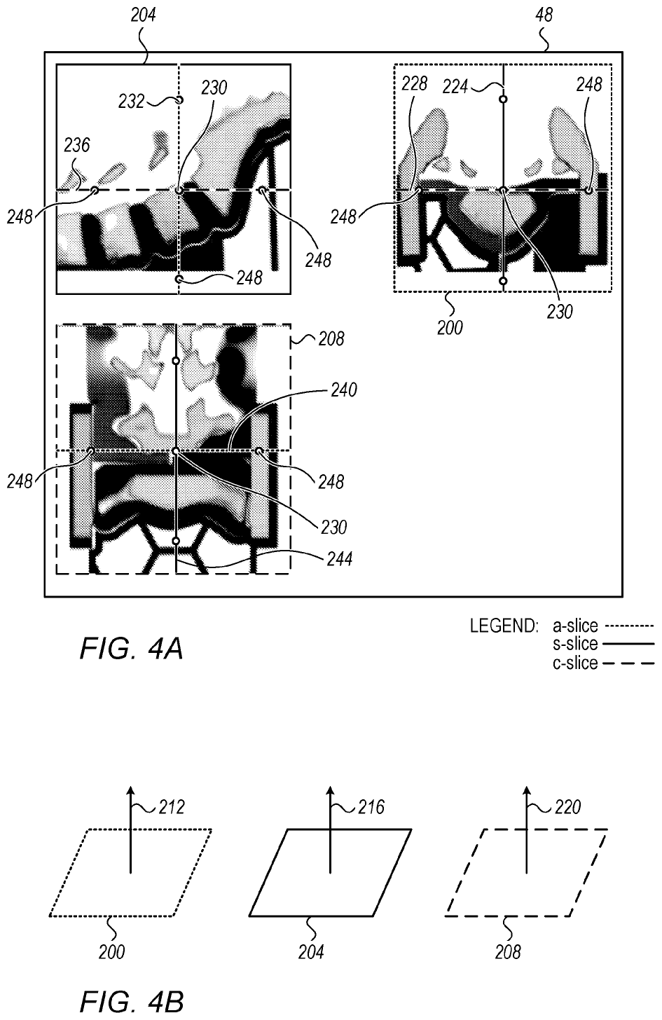

FIG. 3 is a flowchart 100 describing steps performed in the planning stage referred to above, as well as steps performed during the surgical procedure, and FIGS. 4A-8 are schematic drawings illustrating some of the steps, according to an embodiment of the present disclosure.

In an initial step 102 of the planning procedure, comprised in the planning algorithm 52, the professional 22 uses the work station 34 to display images of the anatomy of the patient 30 on the screen 48 of the work station 34. Exemplary schematic drawings of the images as displayed on screen 48 are shown in FIG. 4A.

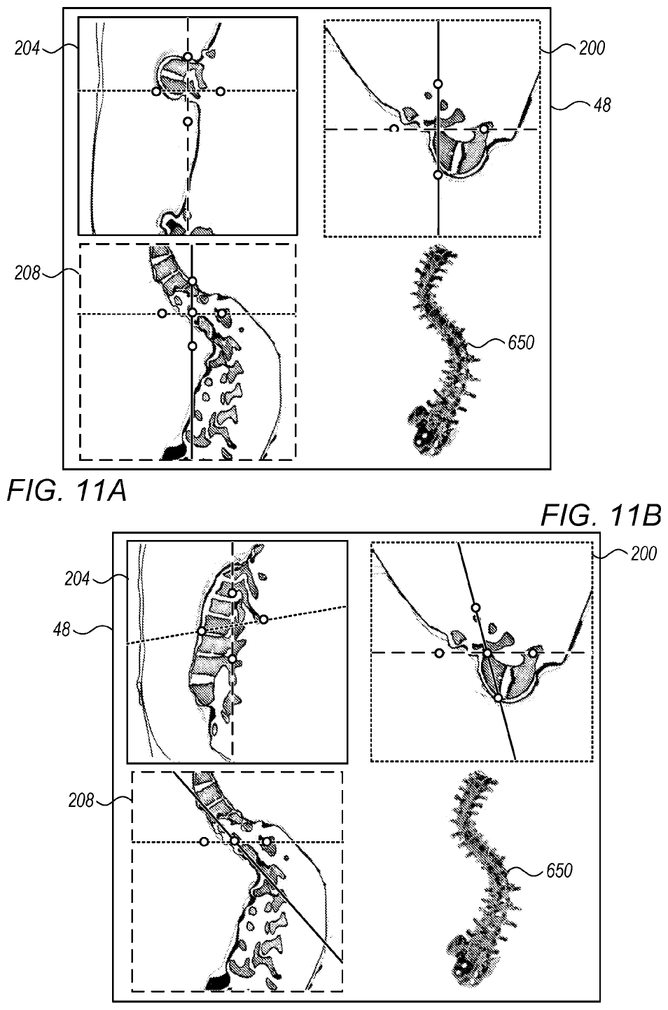

In certain embodiments, the images are generated from a CT scan of the patient 30, for example a DICOM file, that has been previously generated and that is accessed by the professional 22. The images displayed on the screen 48 can be two-dimensional (2D) planes, herein also termed slices, of the scan. In step 102 three 2D slices are displayed in certain embodiments. The parameters of the three 2D slices, i.e., their orientation and position, can be pre-defined by the professional 22, and for simplicity in the following description the three initial 2D slices are assumed to be three mutually orthogonal planes comprising an axial slice 200, a sagittal slice 204, and a coronal slice 208. In some embodiments a three-dimensional image (e.g., a model), generated from the CT scan, is also displayed on screen 48. An example of such a 3D image is provided in an image 650 of FIG. 11A.

As is explained below, during the planning procedure each of the three slices may be individually, separately or independently of the other slices translated and/or rotated from its initial position and orientation. Consequently, rather than using the terms axial, sagittal, and coronal, the three axial, sagittal, and coronal slices are herein respectively termed a-slice 200, s-slice 204, and c-slice 208. In certain embodiments, on the screen 48 the three slices may be differentiated and identified by being framed by different colors; in the figure different lines can be used for the slice frames to identify the slices. The different lines, and the corresponding slices, are shown in a legend of the figure.

As shown in FIG. 4B, each slice has a normal, centered on the slice and orthogonal to it. Thus a-slice 200 has an a-normal 212; s-slice 204 has an s-normal 216; and c-slice 208 has a c-normal 220.

In certain embodiments, each of the three slices is intersected by the other two slices, and in each of the slices the lines of intersection are displayed. The lines of intersection may also be shown on the 3D images. On the screen 48 the lines of intersection can be assigned the color corresponding to the intersecting slice; in the figure the lines of intersection can be identified by the lines of the legend. Thus, a-slice 200 can be intersected by s-slice 204 at an s-intersection line 224, and is intersected by c-slice 208 at a c-intersection line 228. Similarly, s-slice 204 can be intersected by a-slice 200 at an a-intersection line 232, and can be intersected by c-slice 208 at a c-intersection line 236; and c-slice 208 can be intersected by a-slice 200 at an a-intersection line 240, and can be intersected by s-slice 204 at an s-intersection line 244.

It will be understood that any two slices intersect in a straight line, so that the intersection is visible in images of both slices. Furthermore, in certain embodiments, all three slices intersect at one point. herein termed a common intersection point 230. and this point is visible on all three slices.

In certain embodiments, each of the intersection lines has “handles 248,” shown in the figures as solid circles on the lines. Selection by the professional 22 of one or both handles 248 of a given intersection line allows the professional 22 to translate and/or rotate the slice of the selected handle 248. The translation is in any direction selected by the professional 22, that is parallel to the viewed slice. The rotation is by any angle selected by the professional 22, that is around the normal to the viewed slice. Other methods for translating and rotating the slices will be familiar to those having ordinary skill in the art, and all such methods are assumed to be comprised within the scope of the present disclosure. One such method comprises entering numerical values for a number of pixels of translation and/or a number of degrees of rotation in the user interface 49 of the screen 48. The numerical values input may be performed, for example, by entering the values or by translating and rotating the intersection lines, e.g., via handles 248.

In a manipulation step 104, the professional 22 adjusts, as necessary, the positions and angles of the displayed slices, based on a procedure to be performed using the augmented reality assembly 24. In certain embodiments, the professional 22 performs the adjustments by inspection of the scanned images presented on the screen 48. Alternatively or additionally, the professional 22 may perform the adjustments using Hounsfield Unit (HU) values of the DICOM scan, and these may be presented to the professional 22 in the user interface 49.

The following description provides detail of actions performed by the professional 22 in step 104, for a number of different procedures.

### 1. Screw Placement

In the procedure, the professional 22 inserts screw 60 (FIG. 1) into a vertebra of the patient 30.

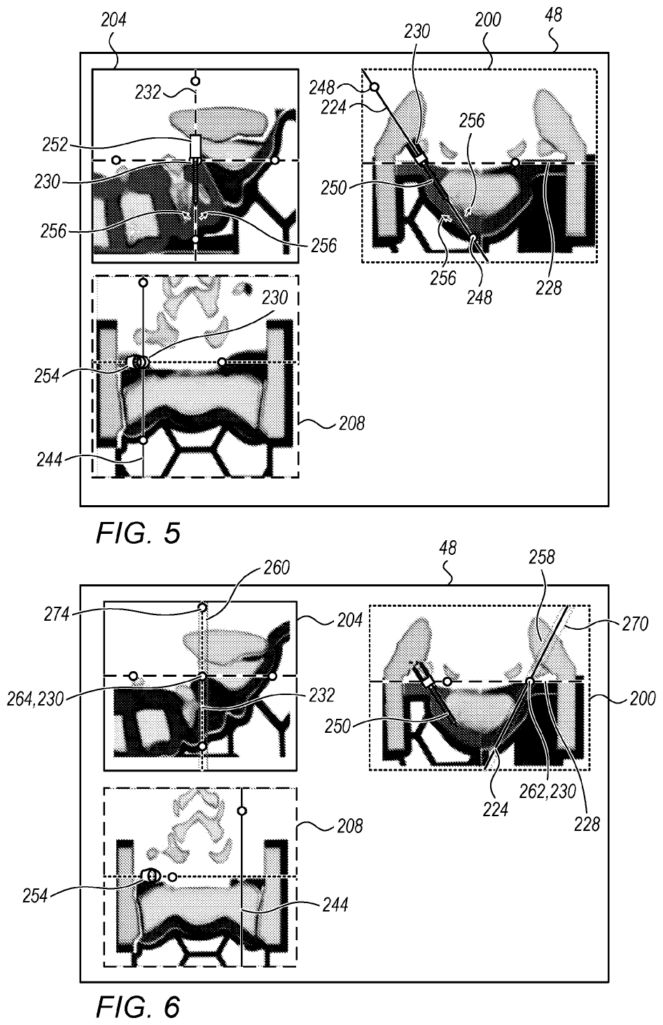

In the planning stage for the procedure, illustrated in FIG. 5 and described hereinbelow, the professional 22 generates an image, using, for example, the processor 56 of the work station 34, showing the screw 60 correctly inserted into the vertebra. During the planning stage the professional 22 may decide to alter the dimensions of the screw 60 from those initially assumed, based on, for example, an inspection of the presented images or a presented plan. For example, professional 22 on inspecting the images, may decide that an initially chosen length of the screw is incorrect. As explained below, the processor 56 is configured to accept such alteration.

As illustrated by s-intersection line 224 for a-slice 200, the professional 22 can rotate s-slice 204 from its initial position (shown in FIG. 4A) by approximately 30° counterclockwise about a-normal 212. The handles 248 of the intersection line 224 can be used to implement the rotation. In certain embodiments, the rotation using the handles 248 is a relatively coarse rotation, and the rotation may be fine-tuned by, for example, using the user interface 49.

S-intersection line 224 for a-slice 200 also illustrates that s-slice 204 has also been translated. Comparing the position of s-intersection line 224 with its initial position shown in FIG. 4A demonstrates that s-slice 204 has been translated parallel to c-intersection line 228. The figure shows that the translation is by approximately 25% of the width of slice 200, to the left of slice 200. The translation, both for coarse and finely-tuned translation, may be implemented in a similar manner to the method for rotation described above. The translation of s-slice 204 is also apparent from the movement of s-intersection line 244 (compared to its initial position in FIG. 4A) in c-slice 208.

The rotation and the translation of the s-slice are such that s-intersection line 224 defines a screw trajectory, desired by the professional, for the placement of screw 60. The result of the rotation and translation of s-slice 204 is illustrated in the different image of the s-slice in FIG. 5 compared to the initial image of the s-slice in FIG. 4A.

Once the s-slice 204 has been translated and rotated so that s-intersection line 224 corresponds to the screw 60 trajectory desired by the professional 22. the professional 22 can add or overlay an icon 250 representing screw 60 on the intersection line, e.g., by activating an “add screw” command. The professional 22 may translate and/or rotate the screw, e.g., by performing these operations on the icon, after the icon has been overlayed. In the illustration, a head of icon 250 has been positioned to be approximately coincident with common intersection point 230 of the three slices.

In certain embodiments, the icon 250 is configured to have dimensions and structure, e.g., length, diameter, shape of screw body, type of screw head, conforming to that of screw 60. As icon 250 is overlayed on s-intersection line 224, the processor 56 generates corresponding icons, as applicable, for s-slice 204 and c-slice 208. Thus, there is a corresponding icon 252 on a-intersection line 232 of s-slice 204. Icon 252 is rotated by 90° about a symmetry axis of the icon, and a head of the icon is approximately coincident with common intersection point 230 of the s-slice.

In certain embodiments, there is a corresponding icon 254, representing the screw head, located at the common intersection point 230 on c-slice 208. In some embodiments, on the addition of icon 250, with the concomitant addition of icon 252, to the slices, processor 56 generates arrows 256 which may be configured, on selection, to permit fine tuning of the position of the icon. Alternatively or additionally, both coarse and fine tuning of the positions of icons 250, 252, and 254, may be accomplished using the user interface 49.

As stated above, during the planning stage the professional 22 may alter the initially assumed dimensions of the screw 60, based on, for example, an inspection of a-slice 200, s-slice 204, and/or c-slice 208. The screw 60 dimensions may be altered via the user interface 49, and the altered dimensions input to the processor 56. In some embodiments, alterations of the screw 60 dimensions are also indicated by alterations of one or more of the icons 250, 252, and 254.

In some embodiments the user may select the screw from a menu and/or add a new screw by entering dimensions. In certain embodiments the screw dimensions may be determined by 3D scanning of a screw, e.g., via a depth sensing method such as structured light. In an embodiment assembly 24 comprises a depth sensing capability, e.g., using structured light, and in this case the assembly may be used to find the dimensions of a screw. For example, cameras 68 of assembly 24 as exemplified in FIG. 2, may be utilized for depth sensing.

On completion of the planning stage hereinabove, i.e., once the professional has defined the screw dimensions and the desired screw trajectory, and positioned the icons of screw 60, processor 56 saves the screw dimensions and the parameters of the screw trajectory, i.e., the orientations of the three slices, and the position of the icons on the trajectory. The saved values may be used for step 106 of flowchart 100, described below.

### 2. Screw Placement and Trajectory Planning

In the procedure, the professional inserts the screw 60 into a vertebra of the patient 30, and, may previously drill a bone of the patient.

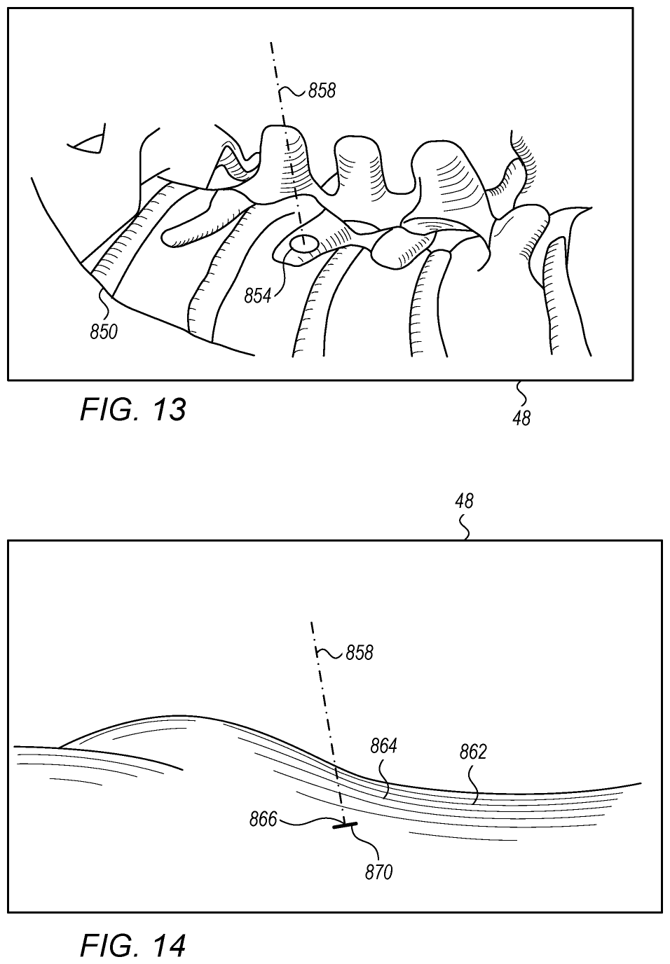

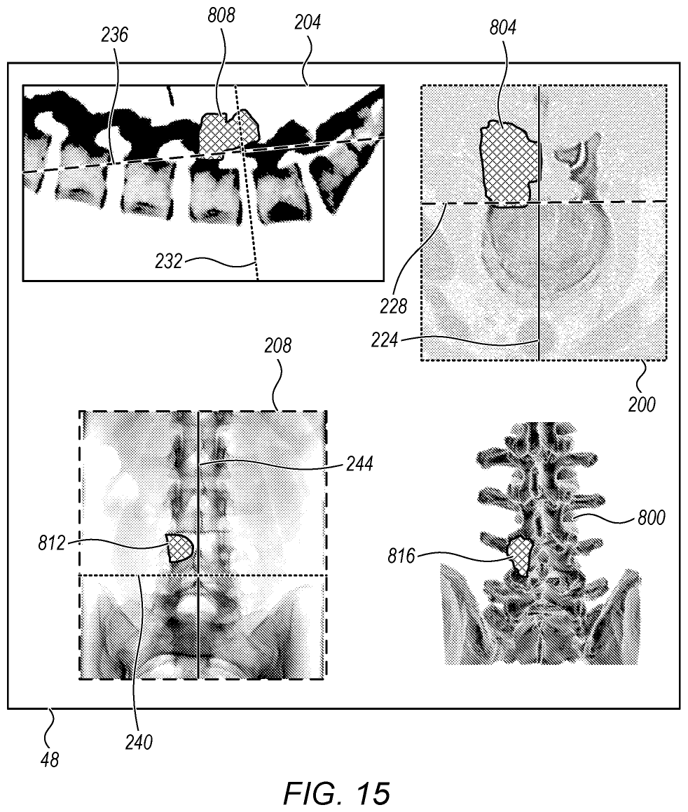

In the planning stage for the procedure, illustrated in FIG. 6, the professional 22 can generate an image, showing the screw correctly inserted into the vertebra and a trajectory, herein also termed an insertion trajectory or a drill trajectory, showing a location of a skin incision and a termination point, to be followed, e.g., by the drill. Location of the skin incision and of a trajectory associated with the incision, as well as parameters associated with the trajectory, and the incision, are described below with reference to FIGS. 13 and 14.

The screw insertion is substantially as described above for Procedure 1 Screw Placement, and the description herein builds on that, and adds material relevant to trajectory planning. Once the parameters of the screw dimensions, the screw trajectory and the screw position have been saved, as described in Procedure 1, professional 22 may re-define, by translation and/or rotation, any of the three slices-a-slice 200, s-slice 204, and c-slice 208-to delineate further parameters for a continuation of the procedure. In the situation illustrated in FIG. 6, professional 22 only moves s-slice 204, so that the images of patient 30 illustrated in a-slice 200 and c-slice 208 are substantially as are illustrated in FIG. 5. Because there is substantially no movement of the two slices, icon 250 and icon 254 are illustrated as respective overlays on a-slice 200 and c-slice 208. (Because s-slice 204 is moved, this is not the case for icon 252, i.e., s-slice 204 does not have as an overlay icon 252, since the screw is not in the plane of the moved slice.)

As illustrated by s-intersection line 224 for a-slice 200, in certain embodiments, the professional rotates s-slice 204 from its initial position (shown in FIG. 4A) by approximately 30° clockwise about a-normal 212. In certain embodiments, s-intersection line 224 is also translated. so that s-slice 204 has translated, parallel to c-intersection line 228, by approximately 25% of the width of slice 200, to the right of slice 200. The translation of s-slice 204 is also apparent in the new position of s-intersection line 244 in c-slice 208.

The translation and rotation described above correspond to the professional defining intersection line 224 as a desired drill direction or trajectory. In an embodiment of the disclosure the professional delineates the desired drill trajectory by overlaying a trajectory icon 258 on intersection line 224. The processor 56 can overlay (e.g., automatically) a corresponding trajectory icon 260 on a-intersection line 232. The two icons typically comprise termination and initial points, corresponding to the intended drill end point and start point in patient 30. The drill start point is herein assumed to comprise a skin incision point in patient 30. As illustrated in FIG. 6, icons 258 and 260 have respective termination points 262, 264 at common intersection point 230. The skin incision point is indicated by initial point 270 for icon 258, and initial point 274 for icon 260. It will be understood that the professional 22 delineates the termination and initial points of the icons by inspection of the scanned images presented on the screen 48. Alternatively or additionally, the professional may delineate the points using HU values of the DICOM scan, so, for example, indicating the skin incision point where the HU value changes from-1000.

The descriptions above describe how different slices may be presented on screen 48. It will be appreciated that views other than those of slices may be generated from the file used to generate the slices, and FIGS. 13 and 14 are schematic illustrations of such other views. FIG. 13 is a schematic 3D view of a section of a patient's spine 850, beneath the skin of the patient, and professional 22 has configured the view to show an intended position 854 for the top of a screw to be placed in the spine. A trajectory 858 associated with the screw placement may be generated and displayed, e.g., automatically or per user (e.g., a medical professional) request. Trajectory 858 may correspond to the path followed for an incision into the patient, so as to access position 854.

FIG. 14 is a schematic 3D view of the back 862 of the patient, i.e., of the patient's skin 864, and it will be appreciated that such a view may be generated, e.g., from the patients file, since the boundaries of the skin with air are apparent from the different Hounsfield Units of skin and air. The view exemplified in FIG. 14 may also be termed a “skin on” view. Trajectory 858 is also shown in the figure, and an intersection 866 of the trajectory with skin 864, corresponding to the position where professional 22 may make an incision in the skin, is shown.

Professional 22 may depict an incision mark 870 or plan an incision path 870 on skin 864. The professional 22 may select parameters for the incision, e.g. length of the incision and/or an orientation of the incision and/or draw on the display using I/O devices such as mouse, touchscreen and the like. Processor 26 may automatically translate a drawn incision and/or the selected orientation into length and angles made by the incision with axes of the patient, correspondingly. A virtual ruler may be generated and displayed to allow the professional to measure the length of a drawn or generated incision path. In certain embodiments, the 2D slice views, such as shown in FIG. 5 or 6, may be also displayed on the screen adjacent to 3D view such as shown in FIG. 13 and “skin-on” view, such as shown in FIG. 14. The slice views may show different views of screw 854 placement which may be used for the incision planning. In certain embodiments, the slices intersection lines may be displayed on the three-dimensional view, such as FIG. 13, and/or the skin-on view, such as FIG. 14.

On completion of this planning stage, in addition to saving the parameters of procedure 1, processor 56 saves the parameters of the insertion or drill trajectory, as well as the parameters of the insertion or drill trajectory icons. Parameters associated with the incision described above with reference to FIGS. 13 and 14 may also be saved by processor 56. The saved parameters are used for step 106 of flowchart 100.

### 3. Manual Multiple Screw Placement and Rod Calculation

In the procedure, the professional inserts screw 60 and further screws, herein by way of example assumed to be two further screws, into respective vertebrae of patient 30. The professional then connects the heads of the multiple screws together by a rod.

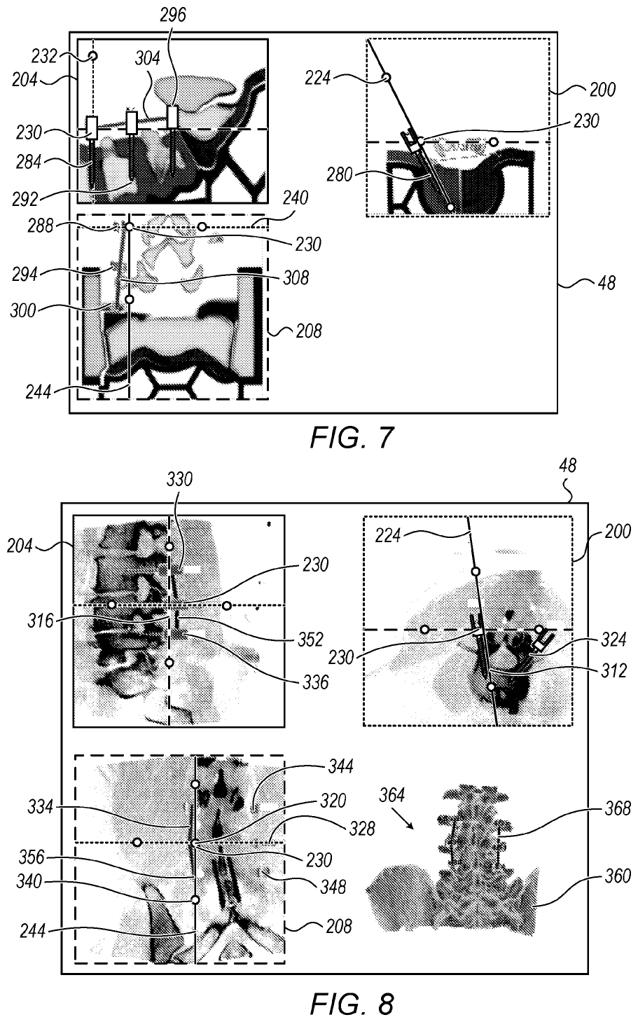

In the planning stage for the procedure, illustrated in FIG. 7, the professional generates an image, showing all the screws inserted into respective vertebrae. The image shows a rod connecting the screw heads, as calculated by the processor. The processor also provides the required length of the rod. The following description of the planning stage assumes, for simplicity, that screw 60, with its initial dimensions, is to be used, and that the dimensions of the other screws are as those for screw 60. The dimensions of screw 60, as well as those of the other screws. may be altered, as described above for procedure 1.

As illustrated by s-intersection line 224 for a-slice 200, and s-intersection line 244 for c-slice 208, s-slice 204 has been rotated by approximately 30° counterclockwise and has also been translated to the left by approximately 25% of the width of the a-slice. In addition, a-slice 200 has been translated, as shown by the leftwards translation of a-intersection line 232 in s-slice 204, and the upwards translation of a-intersection line 240 in c-slice 208.

An icon 280 in a-slice 200 illustrates the placement of screw 60, which in this case has a trajectory corresponding to s-intersection line 224. The head of the screw has been positioned to correspond to common intersection point 230. Once icon 280 has been positioned as describe, processor 56 can position (e.g., automatically) an icon 284 in s-slice 204, and an icon 288 in c-slice 208. Icons 284 and 288 also illustrate that the head of screw 60 is at common intersection point 230.

In addition to the icons for screw 60, in certain embodiments, there are two other sets of icons, for screws in other vertebrae of patient 30. Icons 292 and 296 in s-slice 204 are for two screws in vertebrae proximate to the initial vertebra, one adjacent to the initial vertebra, and another once removed therefrom. Once these icons have been positioned in s-slice 204 by professional 22, the processor 56 can position the corresponding icons 294 and 300 in c-slice 208.

In certain embodiments, multiple screws, as are indicated here, are connected by a rod. In certain embodiments, the processor 56 is configured to present, either automatically or as requested by the professional, respective icons 304 and 308, in s-slice 204 and c-slice 208, representing the rod. The processor may also indicate, by any convenient method, for example in the user interface 49, a length of the rod.

In certain embodiments, the processor 56 saves the parameters of all of the screws, i.e., their trajectories and dimensions, together with the length of the rod connecting the screws. The saved parameters may be used for step 106 of flowchart 100, described below.

### 4. Automatic Multiple Screw Placement

Procedure 3 (described above) explains how icons for multiple screws may be manually positioned by the professional 22. Manual positioning is time consuming, so that procedure 4, described hereinbelow, explains how the positioning preparation may be automated.

In the procedure, the professional inserts a pair of screws on either side of an initial selected vertebra. Further pairs of screws, herein by way of example two further pairs of screws, are inserted into sides of vertebra proximate to the initial vertebra. After the screws have been inserted, the professional connects the screws on a left side of the spinal column with a first rod, and the screws on the right side of the spinal column with a second rod.

In the planning stage for the procedure, illustrated in FIG. 8, the professional can position an initial pair of screw icons on either side of one vertebra. The processor 56 can position (e.g., automatically) subsequent pairs of screw icons on vertebrae selected by the professional 22. Except for being translated to the selected vertebrae, the subsequent pairs have the same parameters, i.e., length, position relative to the vertebra. and orientation, as the initial pair. The processor can also automatically display icons of rods connecting the screw icons, and calculates lengths and, where necessary, angles of bends of the rod icons.

As illustrated by s-intersection line 224 of a-slice 200, and s-intersection line 244 of c-slice 208, s-slice 204 has been rotated by approximately 10° clockwise and has also been translated to the right by approximately 10% of the width of the a-slice. As shown in a-slice 200, professional 22 positions a first screw icon 312 to the left of a selected vertebra, the icon having a trajectory corresponding to s-intersection line 224 and a head at common intersection point 230. The processor automatically generates corresponding screw icons 316 and 320 respectively for s-slice 204 and c-slice 208.

In a-slice 200, prior to positioning screw icon 312, the professional has positioned a second screw icon 324 to the right of the selected vertebra, and the processor has automatically generated a corresponding second screw icon 328 in c-slice 208. The icons on the left of the vertebra may be differentiated from those on the right of the vertebra on screen 48, e.g., by having different colors.

Once professional 22 has positioned a pair of screw icons on either side of the selected vertebra, the professional may select further vertebrae to be populated in a similar manner to those of the selected vertebra. Herein the professional has selected. by way of example, the vertebrae immediately adjacent the selected vertebra, i.e., one vertebra above and one below the selected vertebra. On selection of the further vertebrae, processor 56 automatically generates corresponding screw icons to the left and right of further vertebrae.

The automatically generated icons are: left screw icons 330 and 334 for the upper vertebra, and left screw icons 336 and 340 for the lower vertebra. Visible in c-slice 208 are previous automatically generated right screw icon 344 for the upper vertebra and right screw icon 348 for the lower vertebra.

In some embodiments, except for being translated to the positions of the further vertebrae, the automatically generated screw icons have the same parameters, e.g., orientation and screw length, as the screw icons positioned by the professional. In some embodiments, the automatically generated screw icons have different parameters, e.g., length and/or width, from the screw icons positioned by the professional.

In addition to automatically calculating parameters for, and positioning, the further screw icons, processor 56 is configured to calculate parameters for, and display corresponding icons, for a left-side rod connecting the heads of the left screw icons and a right-side rod connecting the heads of the second screw icons.

S-slice 204 and c-slice 208 respectively show left-side rod icons 352 and 356. As is illustrated, the left-side rod icon has a bend of approximately 10° at its center. Not illustrated in any of the slices, but illustrated in a three-dimensional (3D) representation 360 of the patient's spine, is an image depicting the vertebrae, the inserted screws, a left-side rod image 364, and a right-side rod image 368.

As stated above, the processor 56 calculates parameters for the connecting rods, i.e., the length of each rod and any bends that are necessary in the rods. These parameters, together with parameters of the screws they are connecting, are saved by the processor 56 and may be presented to professional 22 in any convenient form, for example in the user interface 49.

The saved parameters may be used for step 106 of flowchart 100, described hereinbelow.

### 5. Bone Cutting

Procedures 1-4 above describe various procedures that embodiments of the disclosure may be used for, inter alia, working with screws and elements associated with screws. As described herein, a disclosed embodiment of the disclosure may also be used for a bone cutting procedure.

In the procedure, the professional alters the structure of a bone of a patient, for example by bone cutting, bone removal, or bone sculping.

In the planning stage for the procedure, illustrated in FIG. 15, an image of the bones of the patient is generated, including the bone to be worked on, on screen 48. By way of example, the bone to be worked on is assumed to comprise a facet joint of the spine of the patient. As illustrated in the figure, the professional has generated a-slice 200, s-slice 204, and c-slice 208, and the positions of a-intersection line 232 and c-intersection 236, and a-intersection line 240 and s-intersection line 244 are also shown.

A 3D image 800 of the spine of the patient is also shown on screen 48.

The professional has marked on a-slice 200, s-slice 204, and/or c-slice 208, regions of a facet of the spine to be worked on, and these are shown as regions 804, 808, and 812 of the respective slices. As the professional marks a region in a given slice, processor 56 automatically calculates, and as necessary marks, regions for the other slices. In addition, the processor automatically marks, on 3D image 800, a 3D image 816 of the work to be done.

The markings of the work to be done may be by the professional marking a plane on a given slice, using an intersection line with the given slice, to simulate cutting of a bone. Alternatively, the professional may mark a line on a given slice indicating where a cut is to be made. Further alternatively, the professional may mark free contours on any of the slices to indicate where bone is to be cut.

Once the professional has marked up the bones to be worked on, processor 56 is configured to be able to change the 3D image of the bones, according to the mark up, so as to simulate the patient bones after they have been worked on. As stated above the work may be cutting, removal, or sculping, and in all cases processor 56 may generate new 3D images of the bones that the professional can review.

Processor 56 saves the parameters of the slices, together with parameters defining regions to be worked on, herein comprising regions 804, 808, 812, and 816.

The saved parameters may be used, for example, for step 106 of flowchart 100, described hereinbelow.

Returning to flowchart 100 (FIG. 3), in a conversion step 106 of flowchart 100, processor 56 may use, in some embodiments, the saved screw, trajectory, rod and bone cutting parameters, of the planned procedure, to form and save a 3D image of the procedure, including, in the case of the bone cutting, a 3D image of the changed bones, i.e., a 3D image of the bones after they have been worked on. The saved 3D image is made available to the processor 26 of the augmented reality assembly 24, by, for example, being saved in the database 38.

An optional operational step 108 of the flowchart is implemented during an actual procedure on patient 30 which includes using an augmented reality assembly. During the procedure, as illustrated in FIG. 1, professional 22 wears the augmented reality assembly 24. Step 108 is typically implemented when professional 22 is at a stage in the procedure corresponding to the saved image situation of step 106, and is effected by the professional causing processor 26 to present the 3D image on the displays 80. Because, as described above, the processor 26 is tracking patient 30, the processor is able to present the saved 3D image on display 80 correctly aligned, and typically overlayed, with the professional's view of the patient, so as to assist the professional in performing the procedure. In certain embodiments, the 3D image may be displayed on the display area of displays 80 such that it is aligned with the patient anatomy and/or other elements (e.g., a surgery tool or a screw) as viewed by the professional through the portion of each display 80 surrounding the display area.

In certain embodiments, a surgery tool may include a tool marker which may be tracked by a tracking system of system 20. A screw, for example. typically has known dimension and is attached to a tip of a tool. which also has known dimensions, when inserted.

In some embodiments. 2D images of the planning, e.g., as shown in FIGS. 4A-8, may be saved and displayed instead of the 3D image or in addition to the 3D image. In some embodiments, the 3D images may be displayed while not aligned with the professional's view of the patient, e.g., in a top portion or a side portion of the augmented reality display (e.g., AR display 80 or 720). In some embodiments the 2D images and/or 3D images may be displayed on a display other than the AR display, such as the display 48 of the work station 34 instead of or in addition to the display on the AR head-mounted display.

While some of the planning described for the procedures above is partially automated, the time spent by professional 22 is still significant. In the planning stage described hereinbelow, one or more artificial neural network (ANN) are utilized to further automate planning of screws and optionally rods placement.

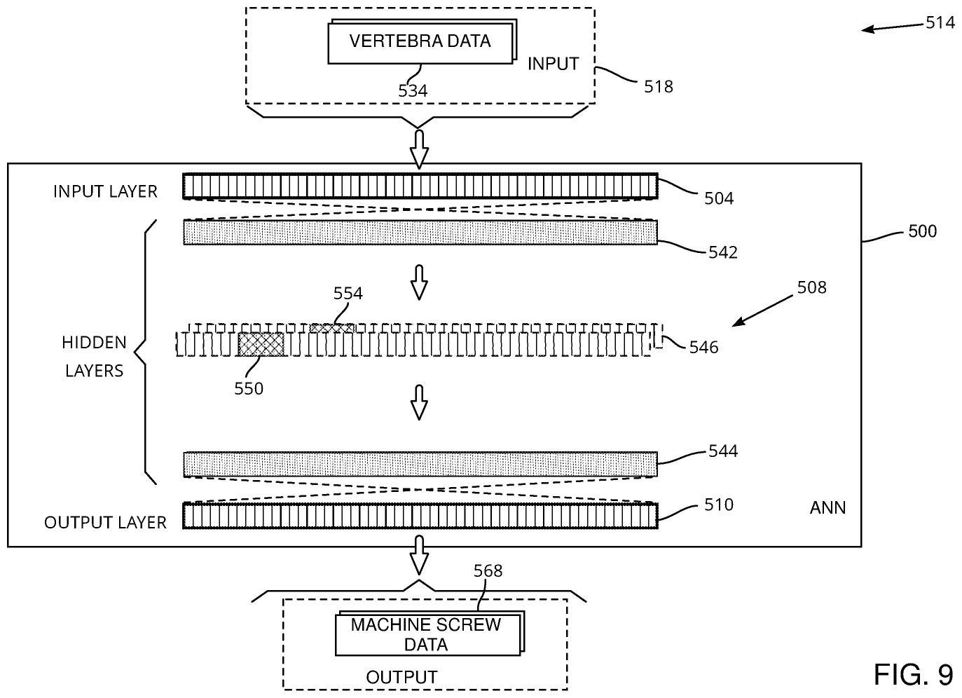

FIG. 9 is a schematic block diagram illustrating the overall structure and operation of an ANN 500. It will be understood that the structure and operation presented here are by way of example, and those having ordinary skill in the art will be aware of networks, having other structures and operations, that function in a similar manner to ANN 500. All such networks are assumed to be comprised within the scope of the present disclosure.

ANN 500 is formed of layers of artificial neurons, and hereinbelow each of the layers is assumed to comprise rectified linear unit (ReLU) neurons. However, the layers of ANN 500 may be comprised of other neurons, such as derivations of ReLU neurons, tanh neurons and/or sigmoid neurons, and those having ordinary skill in the art will be able to adapt the disclosure, mutatis mutandis, for layers with other such neurons.

ANN 500 has a first input layer 504, which is followed by a number of hidden layers 508, and the hidden layers are followed by an output layer 510. These layers are described in more detail below.

In a disclosed example of ANN 500, input layer 504 has a number of neurons corresponding to the number of data elements in an input set of data 514. As described below with reference to flowchart 600, and as illustrated in FIG. 9, a corpus 518 of data sets 514 is used to train ANN 500.

ANN 500 may refer to an untrained ANN or to a trained ANN, hereinbelow termed “inference 500”.

At a training phase, data sets 514 of the corpus may include data derived from one of the screw placement procedures described above. The data may comprise patient scan image data in a “raw” image file. or segmented image data. The patient scan image data typically comprises a CT scan or DICOM file imaging a patient spine. The data may comprise sets of patient spine scan image data comprising a scan performed before screw and/or rod placement and after such were placed. The screws and/or rods may be automatically or manually segmented or otherwise indicated in the post-procedure scans. Additionally, or alternatively, masks or other indications for screws and/or rods may be manually added to spine scan image data to generate simulations of post-procedure scans. For example, a software tool may be used by, e.g., medical professionals, to place virtual indication of screws and/or rods on the spine scan image data. The indicated or labeled data may be used as ground truth for the training of ANN 500.

ANN 500 may be then trained iteratively with the sets or pairs of two images: pre-procedure scan and post-procedure scan. The training may be performed only with respect to screws placement or with respect to screws and rods placement.

In certain embodiments, the input pre-procedure scan may be a cropped image from the scan of the vertebra to be placed with a screw or a cropped image of a set of vertebrae to be placed with screws or with screws and a rod. In certain embodiments, the input scan may be a scan of the spine comprising an indication or marking (e.g., via a mask) of the vertebra or of vertebrae to be placed with screws or with screws and a rod.

The selection, indication or marking of the vertebra or of vertebrae to be placed with screws may be done manually or automatically, e.g., via a segmentation ANN. If a segmentation ANN is used, the entire spine portion shown in the scan may be segmented by the segmenting ANN while the vertebra or the vertebrae to be placed with screws may be manually selected or marked. Those having ordinary skill in the art will be able to use known segmentation networks for the task. Networks of this sort are also described, for example, in U.S. provisional 63/389,958, incorporated herein by reference.

In certain embodiments, multiple ANNs may be trained for different anatomical areas of the spine. Different anatomical areas may be characterized by placement of different types of screws (e.g., having different lengths and different diameters). For example, the lumbar area of the spine may require screws having larger length and diameter than the thoracic area of the spine. Different type of screws may be also used in a specific anatomical area and depending on the anatomic structure of the specific patient. Thus, each anatomical area may be characterized, e.g., by typical range of screw lengths and/or screw diameters. Different anatomical areas may be also characterized by vertebra area of screw placement. For example, in the cervical portion of the spine, screws may be placed in the lateral mass while in other portions of the spine, screws may be typically placed int the center of the pedicle. In certain embodiments, an ANN is trained for the cervical vertebrae, the thoracic vertebrae, the lumbar vertebrae. The sacrum and/or the ilium.

In certain embodiments, multiple ANNs may be trained for different procedures or different type of procedures which may affect the type of screws used and/or manner of placement. Hidden layers 508 are formed as a plurality of parallel sets of layers each set typically comprising at least one convolution layer and/or one fully connected layer. ANN 500 is illustrated as having two fully connected layers 542. 544, with layer 542 following input layer 504 and layer 544 preceding output layer 510. One convolutional layer 546 is shown in FIG. 9. It will be appreciated that the depiction of ANN 500 as having two fully connected layers and one convolutional layer is purely illustrative, and that in practice ANN 500 may have a plurality of fully connected layers. substantially similar to layer 542 and/or a plurality of convolutional layers substantially similar to layer 546.

In the illustrated example convolution layer 546, which consists of at least one filter, or kernel, is configured to perform the convolution of the layer by scanning across the values derived from input layer 504 or from another previous hidden layer. Each kernel is a filter, and the illustrated example depicts layer 546 comprising a first kernel 550 and a second kernel 554. Also, while the illustration shows that the convolution layer has two kernels, there are typically more than two kernels.

Kernels in a convolution layer, such as in layer 546. are typically configured, by their convolution operation, to filter or isolate a feature of the data being analyzed. The kernels operate by sliding, in a step manner with a preset stride, along a presented set of data, and forming convolutions of the section of data “covered” by the kernel after each step.

As stated above, the depiction of ANN 500 is illustrative, and typically there is a multiplicity of convolutional layers similar to layer 546. The network, for example, may include down-sampling and up-sampling layers like max pooling.

In an illustrated disclosed example, output layer 510 is preceded by a fully connected layer 544. However, in certain embodiments ANN 500 may not include a fully connected layer. The sizes of the layers typically being selected to correspond to the size of the data output by ANN 500.

ANN 500 is trained so that its data output comprises machine vertebra data 568. Machine vertebra data 568 comprises a suggestion for screw and/or rod dimensions and indication for screw or screws placement and/or rod placement (e.g., mask or virtual screw and/or rod image overlaid on the scan). It should be noted that the rod typically connects screws placed in multiple vertebrae on one side of the spinal cord. The rod data may comprise, length, diameter and/or bend degree or measure.

Now referring to inference 500, the output may comprise such data with respect to each vertebra separately or with respect to multiple anatomical regions separately (e.g., via multiple ANNs). Additional non-machine-learning logic, algorithms or techniques may be used to provide a scan or rendering combining all of the output. In case the ANNs are only used to provide screw placement suggestion, an additional non-machine-learning logic, algorithm or techniques may be used to provide rod data and/or indication based on the suggested screw placement.

Vertebra data 534 input into inference 500 may comprise a segmented pre procedure scan image of the patient comprising an indication of the vertebra or vertebrae to be placed with screws and/or rods or a cropped image comprising such vertebra or vertebrae.

In a preliminary or initial step, the raw pre procedure scan of the spine or a portion of it may be automatically segmented to vertebrae (by segmenting each vertebra), sacrum and/or ilium. In certain embodiments, the automatic segmentation may be performed by an ANN as described hereinabove. The segmented pre procedure scan may be then presented to the professional which may in turn indicate the vertebra or vertebrae to be placed with screws. Alternatively, or additionally, the raw pre procedure image may be displayed to the professional which may manually segment or indicate the vertebra or vertebrae of interest. In certain embodiments. a portion of the scan comprising the indicated vertebra or vertebrae may be cropped. In certain embodiments, the professional may input data with respect to the specific procedure, e.g., anatomical portion of the spine in which the procedure is performed and/or type of procedure to be performed. Alternatively, or additionally, such data may be automatically obtained from additional data provided with the pre procedure scan. The professional may confirm the correctness of such data. In case multiple ANNs are used, such data allows using the appropriate inference or inferences 500, in case multiple inference 500 are required, e.g., in case screw placement is required in multiple different anatomical portions of the spine. In certain embodiments, the professional may select if to receive only screw placement suggestion or screws and rod placement suggestion.

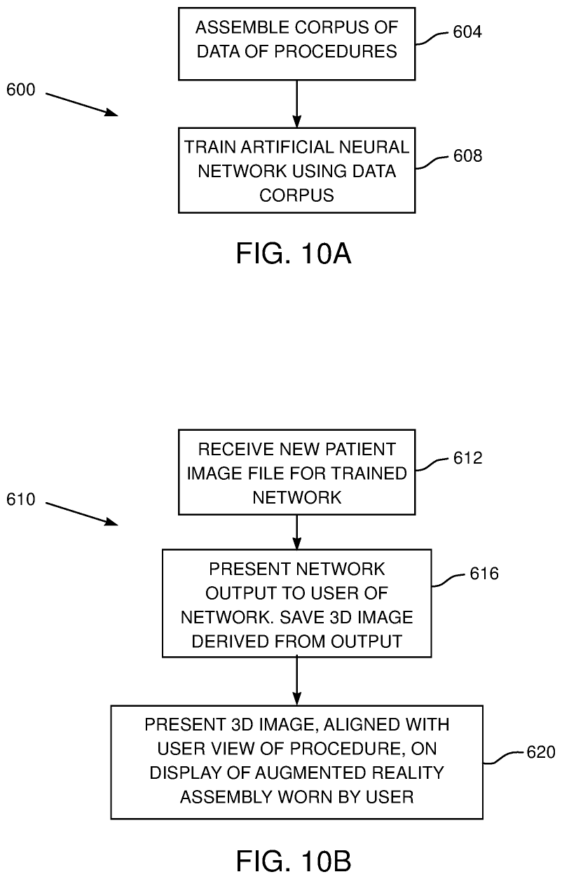

FIG. 10A is a flowchart 600 of steps describing how ANN 500 is trained, and FIG. 10B is a flowchart 610 of steps describing how the trained network is used in certain embodiments.

Referring to flowchart 600 of FIG. 10A, in an initial step 604 of a training stage for the ANN, a corpus of data sets that is to be used for the training is assembled. Details of the data sets, which, e.g., may be derived from spinal procedures, are described above with reference to the description of ANN 500 (FIG. 9).

In a training step 608 the data corpus is used to train the ANN. The training is an iterative process, wherein parameters of the network, such as the weights of the network neurons and the numbers and sizes and weights of the filters of the convolutional layers may be adjusted so as to optimize the output of the network. The training is assumed to be performed using processor 56. but any other suitable one or more processors may be used.

The training may comprise iteratively inputting offsets of the data corpus to the ANN, recording the output of the ANN, and comparing the ground truth input data to the output data using a cost function.

The training may use any cost function known in the art, such as a quadratic cost function or a cross-entropy cost function.

Referring to flowchart 610 of FIG. 10B, once ANN 500 has been trained, as described with reference to FIG. 10A, in an operational step 612. the ANN may receive a segmented raw image file, an image file including having an area of interest indicated or a cropped portion of the image file, as described hereinabove. The image file is typically a CT scan file, of a patient upon whom the professional is intending to perform a spinal procedure. In certain embodiments, and initial or preliminary step may be included, as described hereinabove.

In a data presentation step 616, the results from inference 500, derived from machine screw data 568, may be incorporated into the raw image file of the patient. The results may then be presented to professional 22 as a 3D image of the patient. The presentation may be, for example, via the user interface 49, and professional 22 may accept the results as presented. The professional may also adjust the results. for example by changing a suggested screw type or suggested screw placement. The 3D image may then be saved in database 38 of the augmented reality assembly 24. for access by processor 26.

An optional operational step 620, wherein professional 22 wears the augmented reality assembly 24, is substantially as described for step 108 of flowchart 100. Thus, when implementation of step 620 is effected by the professional, processor 26 is able to present the saved 3D image on displays 80 of the assembly correctly aligned, and typically overlayed, with the professional's view of the patient, so as to assist the professional in performing the procedure.

In certain embodiments, vertebra data 534 may comprise a segmented pre-procedure scan. ANN 500 may then output a data including screws and/or rods placed in all vertebras. The professional may be accordingly presented with the a suggestion for screw and/or rod placement for the entire spine or spine portion. The professional may then select which elements, e.g., screws or rods, to remove.

Reference is now made to FIGS. 11A and 11B, which are schematic diagrams of images of a patient presented to a professional. The descriptions above illustrate how the planning algorithm 52 is used to generate material for screw insertion procedures. However, the algorithm may be used to just improve the images presented to the professional 22. FIG. 11A illustrates slices 200, 204, and 208 that are respectively in the standard axial. sagittal, and coronal orientations. A 3D image 650 is also shown in the figure. In FIG. 11B s-slice 204 has been rotated about a-normal 212 and c-normal 220 (FIG. 4B), so that the image presented in a-slice 204 gives an improved view of the spine.

FIG. 12 is a schematic figure illustrating an exemplary head-mounted display (HMD) 700, according to an embodiment of the present disclosure. HMD 700 is worn by professional 22, and may be used in place of the augmented reality assembly 24 (FIG. 2). In certain embodiments, the HMD 700 comprises an optics housing 704 which incorporates an infrared camera 708. In certain embodiments, the housing 704 comprises an infrared transparent window 712, and within the housing, i.e., behind the window, are mounted one or more infrared projectors 716. In certain embodiments, mounted on housing 704 are a pair of augmented reality displays 720, which allow the professional 22 to view entities, such as part or all of the incision made in the patient back 32, through the displays, and which are also configured to present to the professional images that may be received from the assembly processing unit 28 or any other information.

In certain embodiments, the HMD includes a processor 724, mounted in a processor housing 726, which operates elements of the HMD. Processor 724 typically communicates with the assembly processing unit 28 via an antenna 728, although in some embodiments the processor 724 may perform some of the functions performed by the assembly processing unit 28, and in other embodiments may completely replace the assembly processing unit 28.

In certain embodiments, mounted on the front of the HMD 700 is a flashlight 732. The flashlight 732 projects visible spectrum light onto objects so that professional 22 is able to clearly see the objects through displays 720. Elements of the head-mounted display are typically powered by a battery (not shown in the figure) which supplies power to the elements via a battery cable input 736.

In certain embodiments, the HMD 700 is held in place on the head of the professional 22 by a head strap 740, and the professional 22 may adjust the head strap 740 by an adjustment knob 744.

It should be appreciated that the planning system, software and/or tools described hereinabove may be used individually, separately from or independently of the image-guided navigation system described hereinabove, e.g., with respect to FIG. 1, and including augmented reality system or assembly such as augmented reality assembly 24. The output or products of the planning system, software and/or tools described hereinabove may be used, mutatis mutandis, with image-guided navigation systems other than the augmented-reality system and/or assembly described hereinabove, such as the image-guided navigation system described hereinabove, e.g., with respect to FIG. 1. In some embodiments, the system comprises various features that are present as single features (as opposed to multiple features). For example, in one embodiment, the system includes a single processor, a single HMD, a single camera, a single marker, a single display, a single power source, etc. Multiple features or components are provided in alternate embodiments.

It will be appreciated that the embodiments described above are cited by way of example, and that the disclosure is not limited to what has been particularly shown and described hereinabove. Rather, the scope of the disclosure includes both combinations and subcombinations of the various features described hereinabove, as well as variations and modifications thereof which would occur to persons skilled in the art upon reading the foregoing description and which are not disclosed in the prior art.

We claim:

1. A method comprising:
accessing a computerized tomography (CT) scan of a human subject;
defining a first two-dimensional (2D) slice of the scan and a second 2D slice of the scan, so that the first and the second slices intersect in an intersection line defining a desired trajectory;
overlaying on the intersection line an icon representing an object used in a procedure on the human subject; and
rendering a three-dimensional (3D) image of the human subject, incorporating the overlayed icon, from the CT scan.

2. The method according to claim 1, wherein the object comprises at least one of a screw and a planned trajectory.

3. The method according to claim 2, wherein the planned trajectory comprises a direction for drilling into the human subject.

4. The method according to claim 2 or 3, wherein the icon of the planned trajectory comprises an icon termination point and an icon initial point respectively corresponding to a planned trajectory termination point and a planned trajectory initial point.

5. The method according to claim 4, wherein the planned trajectory initial point comprises a skin incision point, and wherein the planned trajectory termination point comprises a drill end point.

6. The method according to any one of claim 2. 3 or 5, wherein the icon of the screw comprises an icon termination point and an icon initial point respectively corresponding to a screw tip and a screw head.

7. The method according to claim 1, wherein the object comprises a screw configured to be inserted along the desired trajectory into a selected vertebra of the human subject.

8. The method according to claim 7, wherein the 3D image comprises at least one further icon representing at least one further screw configured to be inserted along respective at least one selected trajectory, parallel to the desired trajectory, into respective at least one vertebrae proximate to the selected vertebra.

9. The method according to claim 8, wherein the 3D image comprises a rod icon representing a rod joining respective heads of the screws and the at least one further screw.

10. The method according to claim 7, wherein the 3D image comprises a further icon representing a further screw configured to be inserted along a selected trajectory, different from the desired trajectory, into the selected vertebra.

11. The method according to claim 1, and further comprising, during the procedure, presenting the 3D image on an augmented reality display, while aligning the 3D image with a view through the display of the human subject and the object.

12. A method comprising:
assembling a corpus of data sets of respective procedures performed on human subjects, each data set comprising, for a given human subject, a computerized tomography (CT) scan thereof, an identification of a vertebra therein wherein at least one screw has been inserted, and data descriptive of the at least one screw;
training an artificial neural network (ANN) using the corpus of data sets;
receiving a further CT scan from a further human subject for the trained ANN; and
rendering a three-dimensional (3D) image of the further human subject, in response to an output of the trained ANN, the 3D image comprising a representation of a further human subject vertebra and of a further screw inserted therein.

13. The method according to claim 12, wherein the at least one screw comprises a single screw, and wherein each data set comprises a further identification of at least one further vertebrae, wherein at least one further screw has been inserted, and further data descriptive of the at least one further screw, and a rod identification of a rod joining the single screw and the at least one further screw,
wherein the 3D image comprises, in response to the output of the trained ANN, a rod representation of a rod joining the further screw and at least one additional screw inserted into respective vertebrae of the human subject.

14. Apparatus comprising:
a screen, configured to present a first two-dimensional (2D) slice of a computerized tomography (CT) scan of a human subject and a second 2D slice of the scan, so that the first and the second slices intersect in an intersection line defining a desired trajectory; and
a processor, configured to:
overlay on the intersection line an icon representing an object used in a procedure on the human subject; and
render a three-dimensional (3D) image of the human subject, incorporating the overlayed icon, from the CT scan.

15. The apparatus according to claim 14, wherein the object comprises at least one of a screw and a planned trajectory.

16. The apparatus according to claim 15, wherein the planned trajectory comprises a direction for drilling into the human subject.

17. The apparatus according to claim 15 or 16, wherein the icon of the planned trajectory comprises an icon termination point and an icon initial point respectively corresponding to a planned trajectory termination point and a planned trajectory initial point.

18. The apparatus according to claim 17, wherein the planned trajectory initial point comprises a skin incision point, and wherein the planned trajectory termination point comprises a drill end point.

19. The apparatus according to any one of claim 15, 16 or 18, wherein the icon of the screw comprises an icon termination point and an icon initial point respectively corresponding to a screw tip and a screw head.

20. The apparatus according to claim 14, wherein the object comprises a screw configured to be inserted along the desired trajectory into a selected vertebra of the human subject.

21. The apparatus according to claim 20, wherein the 3D image comprises at least one further icon representing at least one further screw configured to be inserted along respective at least one selected trajectories, parallel to the desired trajectory, into respective at least one vertebrae proximate to the selected vertebra.

22. The apparatus according to claim 21, wherein the 3D image comprises a rod icon representing a rod joining respective heads of the screw and the at least one further screw.

23. The apparatus according to claim 20, wherein the 3D image comprises a further icon representing a further screw configured to be inserted along a selected trajectory, different from the desired trajectory, into the selected vertebra.

24. The apparatus according to claim 14, and further comprising an augmented reality display, and wherein the processor is configured to present the 3D image on the augmented reality display while aligning the 3D image with a view through the display of the human subject and the object.

25. Apparatus comprising:
a display; and
a processor configured to:
assemble a corpus of data sets of respective procedures performed on human subjects, each data set comprising, for a given human subject, a computerized tomography (CT) scan thereof, an identification of a vertebra therein wherein at least one screw has been inserted, and data descriptive of the at least one screw;
train an artificial neural network (ANN) using the corpus of data sets;
input a further CT scan from a further human subject into the trained ANN;
render a three-dimensional (3D) image of the further human subject, in response to an output of the trained ANN, the 3D image comprising a representation of a further human subject vertebra and of a further screw inserted therein; and
present the 3D image on the display.

26. The apparatus according to claim 25, wherein the at least one screw comprises a single screw, and wherein each data set comprises a further identification of at least one further vertebrae, wherein at least one further screw has been inserted, and further data descriptive of the at least one further screw, and a rod identification of a rod joining the single screw and the at least one further screw,
wherein the 3D image comprises, in response to the output of the trained ANN, a rod representation of a rod joining the further screw and at least one additional screw inserted into respective vertebrae of the human subject.

27. The apparatus according to claim 25, wherein the display comprises an augmented reality display, and wherein the processor is configured to present the 3D image on the augmented reality display while aligning the 3D image with a view through the display of the further human subject and the further screw.

28. A method for planning image-guided surgery of a human subject, comprising:
defining a plurality of two-dimensional (2D) slices of a computerized tomography (CT), wherein a first slice and a second slice of the plurality of 2D slices intersect in an intersection line, the intersection line defining a desired trajectory for the image-guided surgery;
overlaying an icon on the intersection line, the icon representing an object used in a procedure on the human subject;
rendering a three-dimensional (3D) image of the human subject from the CT scan that incorporates the icon; and
presenting the 3D image on an augmented reality display, wherein the 3D image is aligned with a view through the augmented reality display of the human subject and the object.

29. The method according to claim 28, wherein the object comprises at least one of a screw and a planned trajectory.

30. The method according to claim 29, wherein the planned trajectory comprises a direction for drilling into the human subject.

31. The method according to claim 29 or 30, wherein the icon of the planned trajectory comprises an icon termination point and an icon initial point respectively corresponding to a planned trajectory termination point and a planned trajectory initial point.

32. The method according to claim 31, wherein the planned trajectory initial point comprises a skin incision point, and wherein the planned trajectory termination point comprises a drill end point.

33. The method according to any one of claim 29, 30 or 32, wherein the icon of the screw comprises an icon termination point and an icon initial point respectively corresponding to a screw tip and a screw head.

34. The method according to claim 28, wherein the object comprises a screw configured to be inserted along the desired trajectory into a selected vertebra of the human subject.

35. The method according to claim 34, wherein the 3D image comprises at least one further icon representing at least one further screw configured to be inserted along respective at least one selected trajectories, parallel to the desired trajectory, into respective at least one vertebrae proximate to the selected vertebra.

36. The method according to claim 35, wherein the 3D image comprises a rod icon representing a rod joining respective heads of the screw and the at least one further screw.

37. The method according to claim 34, wherein the 3D image comprises a further icon representing a further screw configured to be inserted along a selected trajectory, different from the desired trajectory, into the selected vertebra.

38. An apparatus for planning image-guided surgery of a human subject, comprising:
a head mounted display (HMD);
a display configured to present a plurality of two-dimensional (2D) slices of a computerized tomography (CT) scan of the human subject, a first slice and a second slice of the plurality of 2D slices intersecting in an intersection line defining a desired trajectory; and
a processor, configured to:
overlay an icon on the intersection line, the icon representing an object used in a procedure on the human subject;
render a three-dimensional (3D) image of the human subject from the CT scan that incorporates the icon; and
present the 3D image on an augmented reality display, wherein the 3D image is aligned with a view through the augmented reality display of the human subject and the object.

39. The apparatus according to claim 38, wherein the object comprises at least one of a screw and a planned trajectory.

40. The apparatus according to claim 39, wherein the planned trajectory comprises a direction for drilling into the human subject.

41. The apparatus according to claim 39 or 40, wherein the icon of the planned trajectory comprises an icon termination point and an icon initial point respectively corresponding to a planned trajectory termination point and a planned trajectory initial point.

42. The apparatus according to claim 41, wherein the planned trajectory initial point comprises a skin incision point, and wherein the planned trajectory termination point comprises a drill end point.

43. The apparatus according to any one of claim 39, 40 or 42, wherein the icon of the screw comprises an icon termination point and an icon initial point respectively corresponding to a screw tip and a screw head.

44. The apparatus according to claim 38, wherein the object comprises a screw configured to be inserted along the desired trajectory into a selected vertebra of the human subject.

45. The apparatus according to claim 44, wherein the 3D image comprises at least one further icon representing at least one further screw configured to be inserted along respective at least one selected trajectories, parallel to the desired trajectory, into respective at least one vertebrae proximate to the selected vertebra.

46. The apparatus according to claim 45, wherein the 3D image comprises a rod icon representing a rod joining respective heads of the screw and the at least one further screw.

47. The apparatus according to claim 44, wherein the 3D image comprises a further icon representing a further screw configured to be inserted along a selected trajectory, different from the desired trajectory, into the selected vertebra.

48. The apparatus according to claim 38, wherein the augmented reality retaining structure is spectacles.

49. The apparatus according to claim 38, wherein the augmented reality retaining structure is glasses.

50. The apparatus according to claim 38, wherein the augmented reality retaining structure is a head mounted display (HMD).

51. A method comprising:
accessing a three dimensional (3D) anatomy scan of a human subject;
defining a first two-dimensional (2D) view the scan, a second 2D view of the scan, and a third 2D view of the scan to provide an initial view of an area of interest of the human subject, wherein the first, second and third 2D views are generated from the scan; and
rotating at least one of the first, second, and third 2D views so as to provide an improved view of the area of interest.

52. The method according to claim 51, wherein the first, second and third 2D views define respective first, second and third normals thereto, and wherein rotating the at least one of the first, second, and third 2D views comprises rotating the first view about one of the second normal and the third normal.

53. The method according to claim 51, wherein the first, second and third 2D views define respective first, second and third normals thereto, and wherein rotating the at least one of the first, second, and third 2D views comprises rotating the first view about the second normal and the third normal.

54. The method according to any of claim 51, 52 or 53, wherein the 2D views are 2D slices of the scan.

55. The method according to any of claim 51, 52 or 53, wherein the 2D views are axial, sagittal, and coronal views of the human subject.

56. The method according to any of claim 51, 52 or 53, wherein the 3D anatomy scan is a Computerized Tomography (CT) scan.

57. The method according to any of claim 51, 52 or 53, wherein at least one of the first 2D view the scan, the second 2D view of the scan, and the third 2D view of the scan is a digitally reconstructed radiograph (DRR).

58. The method according to any of claim 51, 52 or 53, wherein the method further comprises translating at least one of the first, second, and third 2D views so as to provide an improved view of the area of interest.

59. The method according to any of claim 51, 52 or 53, wherein the rotating is performed following a user instruction.

60. The method according to claim 51, wherein the first, second and third 2D views define respective first, second and third normals, and wherein each 2D view of the first, second, and third 2D views may be rotated with respect to only one other normal of the first, second and third normals.

61. A system as described and illustrated herein.

62. A method as described and illustrated herein.
